(19) United States
(12) Patent Application Publication
(10) Pub. No.: US 2024/0299636 A1
(43) Pub. Date: Sep. 12, 2024

(54) Wearable Breast Pumps

(71) Applicant: Better Babies LLC, South Pasadena, CA (US)

(72) Inventor: Janice Gott, Woodland Hills, CA (US)

(21) Appl. No.: 18/596,279

(22) Filed: Mar. 5, 2024

Related U.S. Application Data
(60) Provisional application No. 63/451,576, filed on Mar. 11, 2023.

(51) Int. Cl. A61M 1/06

(52) U.S. Cl. CPC A61M 1/062; A61M 2209/088

(57) ABSTRACT

Wearable breast pumps optimized for user privacy and wearability are provided. In one embodiment, a wearable breast pump is provided, the wearable breast pump comprising: a flange that is hollow, wherein the flange comprises: a main opening for receiving and attaching to a user's breast; and a first transition opening that allows passage between the flange and a milk reservoir; the milk reservoir that is hollow and connected to the flange, the milk reservoir comprising: a second transition opening that allows passage between the flange and the milk reservoir; a dome surface that drapes a user's clothing when the wearable breast pump is in a pump orientation; and a flat surface connected to the dome surface, wherein the flat surface allows the wearable breast pump to rest horizontally in a storage orientation.

### CROSS-REFERENCE TO RELATED APPLICATION

The current application claims priority to U.S. Provisional Patent Application No. 63/451,576, filed on Mar. 11, 2023, the disclosure of which is incorporated herein by reference.

### FIELD OF THE INVENTION

The present disclosure generally relates to pumps and more specifically to wearable breast pumps.

### BACKGROUND

Breast pumps are mechanical devices that may be used to extract milk from a user's breast. For example, breast pumps may be used for expressing and storing milk which may be later bottle-fed to an infant. Breast pumps may be categorized as manual devices that are powered manually (e.g., by hand) or automatic devices powered by battery or electricity. Breast pumps may also assist in overcoming various challenges such as, but not limited to, difficulties in latching, separation of a mother from an infant, and feeding an infant who cannot yet extract sufficient milk from the breast.

### SUMMARY OF THE INVENTION

The various embodiments of the present wearable breast pumps have several features, no single one of which is solely responsible for their desirable attributes. Without limiting the scope of the present embodiments as expressed by the claims that follow, their more prominent features now will be discussed briefly. After considering this discussion, and particularly after reading the section entitled “Detailed Description,” one will understand how the features of the present embodiments provide the advantages described herein.

In a first aspect, a wearable breast pump optimized for user privacy and wearability is provided, the wearable breast pump comprising: a flange that is hollow, wherein the flange comprises: a main opening for receiving and attaching to a user's breast; and a first transition opening that allows passage between the flange and a milk reservoir; the milk reservoir that is hollow and connected to the flange, the milk reservoir comprising: a second transition opening that allows passage between the flange and the milk reservoir; a dome surface that drapes a user's clothing when the wearable breast pump is in a pump orientation; and a flat surface connected to the dome surface, wherein the flat surface allows the wearable breast pump to rest horizontally in a storage orientation.

In an embodiment of the first aspect, the flange comprises a base that is flat and an apex located opposite the base, wherein the flange tapers from the base towards the apex.

In another embodiment of the first aspect, the flange is a three-dimensional shaped cone with the apex sliced by a truncation plane.

In another embodiment of the first aspect, the truncation plane is offset relative to a vertical plane defined along the base.

In another embodiment of the first aspect, the truncation plane is offset relative to the vertical plane by 15 degrees.

In another embodiment of the first aspect, the milk reservoir is attached to the flange along the truncation plane.

In another embodiment of the first aspect, the milk reservoir further comprises a back surface that runs along the truncation plane.

In another embodiment of the first aspect, the back surface comprises first and second back sides are flat.

In another embodiment of the first aspect, the back surface further comprises a concave portion located between the first and second back sides, wherein the concave portion allows the wearable pump to wear closer to the user's body.

In another embodiment of the first aspect, the back surface tapers in towards the user at a back surface taper angle relative to the vertical plane.

In another embodiment of the first aspect, the back surface taper angle is equal to 15 degrees.

In another embodiment of the first aspect, the flat surface of the milk reservoir tapers towards the user at a flat surface taper angle relative to the vertical plane.

In another embodiment of the first aspect, the flat surface taper angle is greater than the back surface taper angle.

In another embodiment of the first aspect, the flat surface taper angle is at least double the back surface taper angle.

In another embodiment of the first aspect, the flat surface taper angel is 45 degrees.

In another embodiment of the first aspect, the milk reservoir has a milk reservoir diameter measured at a widest portion of the milk reservoir and the flange has a flange diameter measured at a widest portion of the flange, wherein the milk reservoir diameter is greater than the flange diameter.

In another embodiment of the first aspect, the flat surface of the milk reservoir is circular.

In another embodiment of the first aspect, the milk reservoir further comprises a first front side and a second front side, wherein the first and second front sides are curved relative to the flat surface.

In another embodiment of the first aspect, the wearable breast pump further comprises a lid that connects to the base of the flange and seals milk within the wearable pump when the wearable pump is in the storage orientation.

In another embodiment of the first aspect, the milk reservoir further comprises measurement markings that indicate an amount of milk that is within the milk reservoir when the wearable pump is in the storage orientation.

### BRIEF DESCRIPTION OF THE DRAWINGS

FIG. 1A is a right side view of a wearable pump in accordance with certain embodiments of the invention.

FIG. 1B is a front perspective view of a wearable pump in accordance with certain embodiments of the invention.

FIG. 1C is a back perspective view of a wearable pump in accordance with certain embodiments of the invention.

FIG. 1D is a top perspective view of a lid of a wearable pump in accordance with certain embodiments of the invention.

FIG. 2A is a right side view of a wearable pump in a pump orientation in accordance with certain embodiments of the invention.

FIG. 2B is a right side view of a wearable pump in a storage orientation in accordance with certain embodiments of the invention.

FIGS. 3A-C illustrate a process for attaching a wearable pump to a user in accordance with certain embodiments of the invention.

FIG. 4 is a diagram illustrating a conventional pump attached to a user in accordance with the prior art.

FIG. 5 is a diagram illustrating a wearable pump attached to a user in accordance with certain embodiments of the invention.

FIG. 6A is a schematic diagram illustrating a front view of a wearable pump in accordance with certain embodiments of the invention.

FIG. 6B is a schematic diagram illustrating a cross-sectional view of a wearable pump along line A-A of FIG. 6A in accordance with certain embodiments of the invention.

FIG. 6C is a schematic diagram illustrating a back view of a wearable pump in accordance with certain embodiments of the invention.

FIG. 6D is a schematic diagram illustrating a top view of a wearable pump in accordance with certain embodiments of the invention.

FIG. 7A is a schematic diagram illustrating a top view of a lid for a wearable pump in accordance with certain embodiments of the invention.

FIG. 7B is a schematic diagram illustrating a cross-sectional view of a lid for a wearable pump along line A-A of FIG. 7A in accordance with certain embodiments of the invention.

FIG. 7C is a schematic diagram illustrating a bottom view of a lid for a wearable pump in accordance with certain embodiments of the invention.

FIG. 7D is a schematic diagram illustrating a side view of a lid for a wearable pump in accordance with certain embodiments of the invention.

### DETAILED DESCRIPTION OF THE DRAWINGS

The following detailed description describes the present embodiments with reference to the drawings. In the drawings, reference numbers label elements of the present embodiments. These reference numbers are reproduced below in connection with the discussion of the corresponding drawing features.

The embodiments of the present wearable breast pumps (may also be referred to as “wearable pumps”) are described below with reference to the figures. These figures, and their written descriptions, indicate that certain components of the apparatus are formed integrally, and certain other components are formed as separate pieces. Those of ordinary skill in the art will appreciate that components shown and described herein as being formed integrally may in alternative embodiments be formed as separate pieces. Those of ordinary skill in the art will further appreciate that components shown and described herein as being formed as separate pieces may in alternative embodiments be formed integrally. Further, the present wearable pumps are described in the context of wearability with a user's shirt. However, the use of a shirt is merely exemplary and the present wearable pumps may be utilized with various clothing, or without clothing. For example, the present wearable pumps may be used with bras, tee-shirts, tank tops, sweaters, sweatshirts, blouses, dresses, jumpsuit, etc., or without clothing.

One aspect of the present embodiments includes the realization that conventional battery-powered and electric pumps (may be referred to collectively as “electric pumps”) require a motorized pump. Electric pumps may be a convenient option for mothers who need to express milk regularly or who have difficulty with manual pumping. However, electric pumps have various drawbacks such as high cost, noise, power source requirements, cleaning and maintenance, etc. In addition, electric pumps are generally bulky and heavy, making them difficult to transport or use in public places. The present embodiments solve these problems by providing a wearable breast pump that is manually operated and optimized for wearability. The present embodiments may be manufactured at a lower cost, utilized without noise, and provide for easy cleaning and maintenance. Further, the present embodiments are configured to be discrete and wearable under a user's clothing, as further described.

Another aspect of the present embodiments includes the realization that conventional manual pumps are not optimized for user privacy and wearability. For example, conventional manual pumps are not designed to be wearable and discreet while being utilized. Instead, conventional manual pumps are typically designed for manual operations (as opposed to motor-based) but are not created to be worn when the pump is in operation. The present embodiments solve these problems by providing a wearable pump that includes a milk reservoir having a dome surface and a flat surface that is angled from the dome surface towards the user's body that allows a user's clothing to drape naturally over the wearable pump for user privacy and wearability. These and other aspects and advantages of the present embodiments are described in further detail below.

Turning now to the drawings, wearable pumps in accordance with embodiments of the invention are disclosed. In many embodiments, wearable pumps may have a first orientation for pumping (may also be referred to as the “pump orientation”) and a second orientation for storage (may also be referred to as the “storage orientation”). In various embodiments, the wearable pumps may include a flange that allows the wearable pump to attach to a user and a milk reservoir for receiving milk expressed from the user. In several embodiments, the milk reservoir may include a dome surface that allows a user's clothing to drape over the wearable pump when the wearable pump is in the pump orientation, as further described below. In a variety of embodiments, the milk reservoir may also include a flat surface that allows the wearable pump to rest horizontally in the storage orientation. In some embodiments, the flange may have a conical shape (e.g., a right circular cone, an oblique circular cone, etc.) with a region including its apex cut off by a plane (may also be referred to as a “truncation plane”). In various embodiments, the truncation plane is angled relative to a base of the flange, as further described below. In some embodiments, the milk reservoir may include a back surface having a concave portion to further assist with wearability, as further described below. Wearable pumps having flanges and milk reservoirs in accordance with embodiments of the invention are further discussed below.

### Wearable Pumps Having Flanges and Milk Reservoirs

As described herein, the present embodiments optimize for the wearability of the pump. In many embodiments, the wearable pumps include a flange and a milk reservoir configured such that the wearable pump is discrete and concealed under the user's clothing.

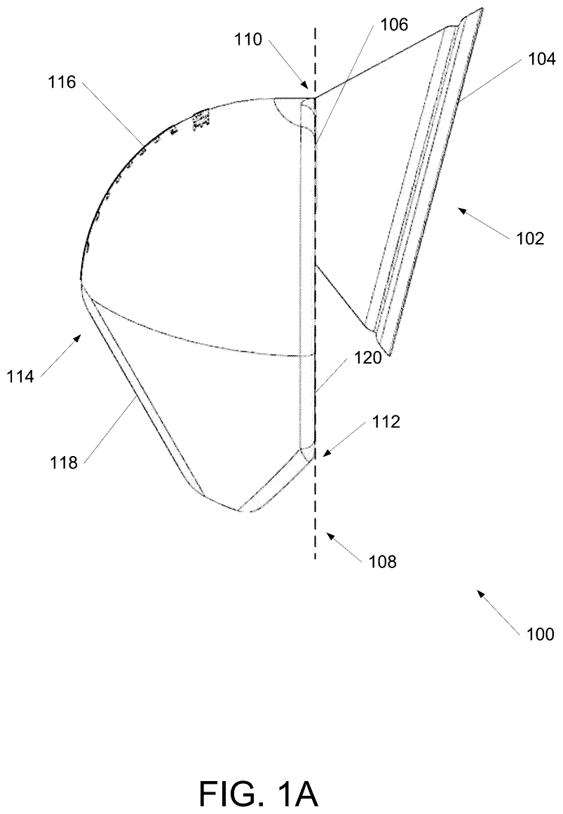

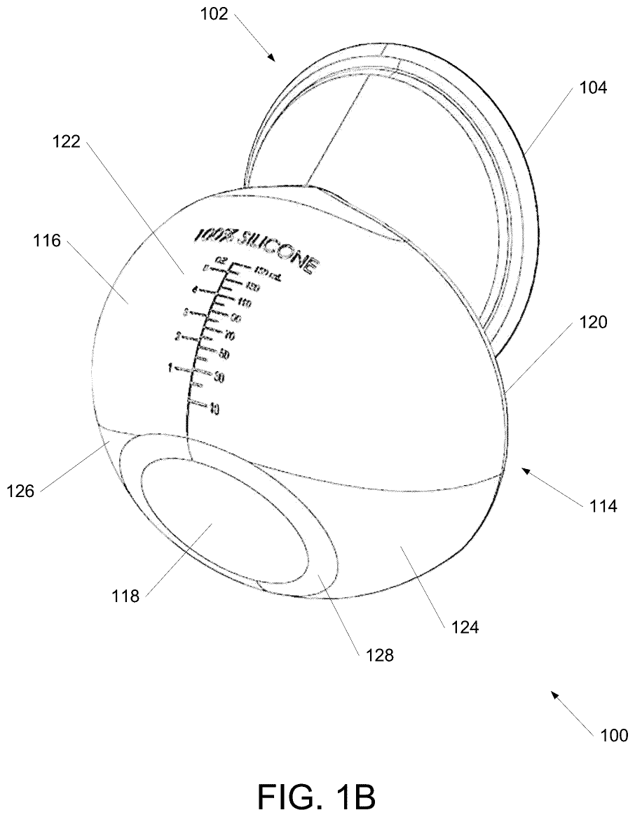

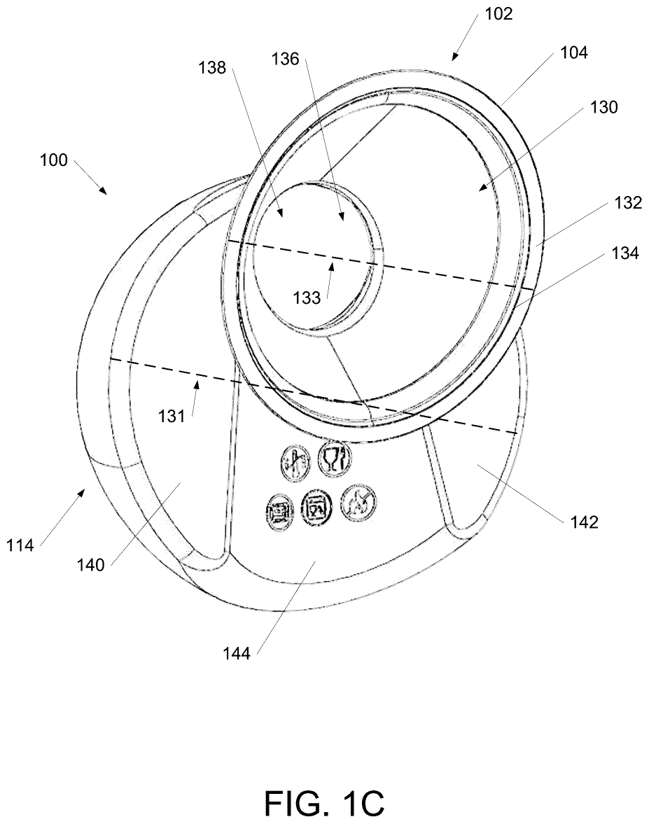

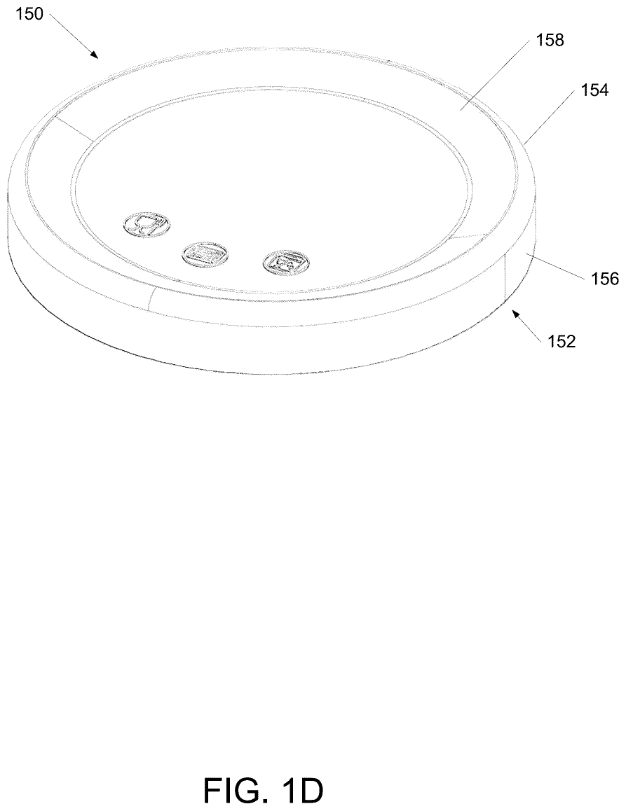

A right side view of a wearable pump in accordance with certain embodiments of the invention is shown in FIG. 1A. The wearable pump 100 may include a flange 102 for attaching to a user. For example, the flange 102 may be hollowed and configured to receive a user's breast for expressing milk. In many embodiments, the flange 102 may be conical shape (i.e., three-dimensional shaped cone) that is flat on one end (may be referred to as a “base” 104) and gradually tapers towards an “apex” 106. However, in various embodiments, the flange 102 may be a three-dimensional shaped cone with a region including its apex 106 cut off (e.g., sliced) by a truncation plane 108. In some embodiments, the truncation plane 108 may be angled relative to the base 104 of the flange 102 such that the truncation plane 108 is closer to the user's body at a bottom end 112 than at a top end 110, and thus the truncation plane 108 may be angled towards the user's body when the truncation plane 108 is extended below the flange 104.

In reference to FIG. 1A, the wearable pump 100 may also include a milk reservoir 114 having a dome surface 116 that allows the user's clothing to drape on the dome surface 116 when the wearable pump 100 is in a pump orientation, as further described below. The milk reservoir 114 may also include a flat surface 118 connected to the dome surface 116, where the flat surface 118 allows the wearable pump 100 to rest horizontally in a storage orientation, as further described below. The milk reservoir 118 may also include a back surface 120 that may include a concave portion, as further described below. In many embodiments, the milk reservoir 114 may be connected to the flange 102 along the truncation plane 108 and the back surface 120 of the milk reservoir 114 may extend along the truncation plane 108 such that the back surface 120 is angled relative to the base 106 of the flange 102, as further described below.

A front perspective view of a wearable pump 100 in accordance with certain embodiments of the invention is shown in FIG. 1B. As described above, the milk reservoir 114 may include a dome surface 116 for allowing a user's clothing to drape when the wearable pump 100 is being used and is in the pump orientation. In addition, the milk reservoir 114 may include a flat surface 118 that allows the wearable pump 100 to store milk in the storage orientation. The dome surface 116 may also include measurement markings 122 that indicates the amount of milk that is in the milk reservoir 114. In many embodiments, the measurement markings 122 may be configured to be used when the wearable pump 100 is in the storage orientation (i.e., the wearable pump 100 is orientated on a flat surface). In some embodiments, the measurement markings 122 may include one or more measurements of volume such as, but not limited to milliliters (i.e., mL) or ounces (i.e., Oz).

In further reference to FIG. 1B, the dome surface 116 may provide a round shape to the milk reservoir 114 allowing the user's clothing to drape more naturally. Further, the milk reservoir 114 may include a first front side 124 and a second front side 126. In some embodiments, the first and second front sides 124, 126 may be curved relative to the flat surface 118. In addition, the flat surface 118 may be a round shape that is flat and the milk reservoir may also include a transition layer 128 around the edge of the flat surface 118. In some embodiments, the transition layer 128 may gradually taper the transition from the flat surface 118 to the surrounding surfaces such as, but not limited to, the first and second front sides 124, 126, and the dome surface 116. As illustrated to FIG. 1B and further described below, the flat surface 118 (and the transition layer 128) may taper towards the user's body when in the pump orientation.

A back perspective view of a wearable pump 100 in accordance with certain embodiments of the invention is shown in FIG. 1C. As described above, the flange 102 may be hollow and configured to attach to the user. For example, the flange 102 may include a main opening 130 for receiving and attaching to a user's breast. In various embodiments, the main opening 130 may be along the base 104 of the flange 102. In many embodiments, the flange 102 may include an outer rim 132 and an inner rim 134 to assist in creating a seal around the breast. Further, the flange 102 may include a transition opening (may also be referred to as a “first transition opening” 136) that allows passage between the flange 102 and the milk reservoir 114.

In reference to FIG. 1C, the milk reservoir 114 may be hollowed and include a corresponding transition opening (may also be referred to as a “second transition opening” 138) that allows passage between the flange and the milk reservoir. For example, the first and second transition openings 136, 138 may allow for at least a portion of a user's nipple to pass into the milk reservoir 114 such that milk is expressed into the milk reservoir 114, as further described below. For example, in operation, the wearable pump 100 may extract milk from the breast by creating a seal around the breast via the flange 102, allowing the nipple to pass into the milk reservoir 114 via the first and second transition openings 136, 138, and applying and releasing suction to the nipple (e.g., when the user squeezes the milk reservoir 114, as further described below), which expresses milk from the breast (each suction and release combination may be referred to as a “cycle.”) Once the wearable pump 100 is attached, as further described below, the user may apply and repeat a cycle by squeezing the milk reservoir 114.

In reference to FIG. 1C, the various configurations of the milk reservoir 114 may allow the user's clothing to drape naturally, as described herein. For example, the milk reservoir 114 may have a diameter (e.g., as measured at the back surface 120 of the milk reservoir 114 and/or measured as the widest portion of the milk reservoir 114) (may also be referred to as a “milk reservoir diameter” 131) that is greater than a diameter the flange 102 (e.g., as measured at the base 104 of the flange 102 and/or measured as the widest portion of the flange 102) (may also be referred to as a “flange diameter” 133). By having a greater diameter, the milk reservoir 114 may be wider and/or flatter than the flange 102, allowing the user's clothing to drape more naturally.

In reference to FIG. 1C, the milk reservoir 114 may include a first back side 140 and a second back side 142. In some embodiments, the first and second back sides 140, 142 may be flat. The milk reservoir 114 may also include a concave portion 144 that may be configured to provide a user additional space so that the wearable pump 100 may only contact the user at the flange 102. In many embodiments, the concave portion 144 (along with the various other features described herein) allow for the wearable pump 100 to be flatter and wear closer to the user resulting in the user's clothing to protrude less than when using conventional pumps. In many embodiments, the back surface 120 may be defined as a plane that is generated along the surfaces of the first and second back sides 140, 142.

A top perspective view of a lid 150 for a wearable pump in accordance with certain embodiments of the invention is shown in FIG. 1D. In some embodiments, a lid 150 may be attached to a flange (e.g., flange 102) when a wearable pump (e.g., wearable pump 100) is in the storage orientation. The lid 150 may include a receiving slot 152 that mates with an outer rim (e.g., outer rim 132) of the flange. For example, the receiving slot 152 may be circular and include a slot 154, a flat edge 156, and a tapered edge 158, as further described below. In some embodiments, the slot 154 may be shaped to receive the outer rim of the flange and apply pressure via the flat edge 156 and the tapered edge 158 to lock the lid 150 onto the flange. By applying the lid 150 to the flange, the wearable pump may seal off the milk inside the milk reservoir to the outside. In many embodiments, the lid 150 may be made using the same materials as the flange (and the milk reservoir) such as, but not limited to, a food grade silicone.

Although wearable pumps are discussed above with respect to FIGS. 1A-D, any of a variety of wearable pumps including wearable pumps having various flanges and/or milk reservoirs as appropriate to the requirements of a specific application can be used in accordance with embodiments of the invention. Pump orientations and storage orientations of wearable pumps in accordance with embodiments of the invention are discussed further below.

### Wearable Pumps Having Pump and Storage Orientations

The present embodiments optimize for the wearability of the wearable pump. As described above, when in a pump orientation, the flange and the milk reservoir are configured such that the wearable pump is discrete and concealed under the user's clothing. When in storage orientation, the flange and the milk reservoir are further configured such that the wearable pump allows for storage of milk.

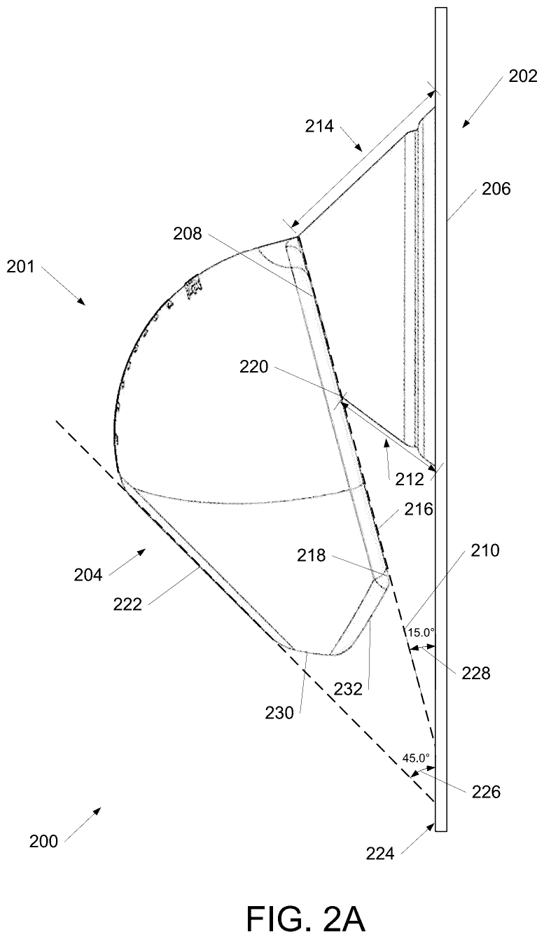

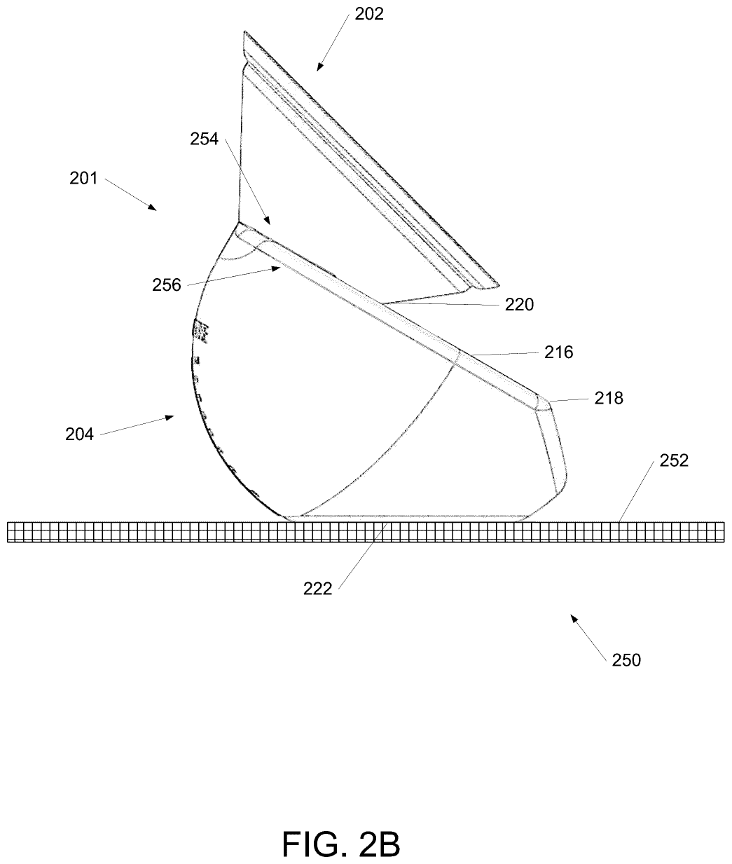

A right side view of a wearable pump 201 in a pump orientation 200 in accordance with certain embodiments of the invention is shown in FIG. 2A. In the pump orientation 200, the flange 202 and the milk reservoir 204 are attached and shaped to conceal the wearable pump under the user's clothing. For example, the flange 202 may have a cone shape having a base side 206 and an apex side 208, where the apex side 208 is truncated by a truncation plane 210 that is angled relative to the base 206 of the flange 202. By utilizing a truncation plane 210 that is angled, the flange 202 has a shorter bottom slant length 212 relative to a top slant length 214.

In reference to FIG. 2A, the milk reservoir 204 may include a back surface 216 and the milk reservoir 204 may be attached to the flange 202 along the truncation plane 210 via the back surface 216 of the milk reservoir 204. As described above, the truncation plan 210 may be angled relative to the base 206 of the flange 202, and thus the back surface 216 of the milk reservoir 204 may also be angled relative to the base 206 of the flange 202. In the pump orientation 200, the base 206 of the flange 202 is vertical (or substantially vertical) and thus the back surface 216 of the milk reservoir 204 (and the truncation plane 210) is angled relative to the user. For example, in the pump orientation 200, the back surface 216 of the milk reservoir 204 may be pointed towards the user at a bottom end 218 of the back surface 216 and pointed away from the user at a top end 220 of the back surface 216 (i.e., tapers in towards the user). In various embodiments, such a configuration allows a greater portion of the milk reservoir 204 to be concealed, thereby increasing wearability.

In further reference to FIG. 2A, the flat surface 222 tapers towards the user at a flat surface taper angle 226 relative to a vertical plane 224, where the vertical plane 224 may be defined as a plane along the base 206 of the flange 202 (i.e., when a plane along the base 206 of the flange 202 is vertical). Further, the back surface 216 tapers in towards the user at a back surface taper angle 228 (e.g., 15 degrees) relative to the vertical plane 224. In many embodiments, the flat surface taper angle 226 (e.g., 45 degrees) is greater than the back surface taper angle 228. In some embodiments, the flat surface 222 and the back surface 216 may be connected with one or more bottom surfaces (e.g., first bottom surface 230 and/or second bottom surface 232). In some embodiments, the flat surface 222 and the back surface 216 may connect directly. In many embodiments, the flat surface and back surface taper angles 226, 228, the length of the flat surface 222, the length of the back surface 216, and/or the one or more bottom surfaces 230, 232 may be adjusted to adjust for a center of gravity of the wearable pump 200 when filled with milk. By adjusting the center of gravity of the wearable pump 200, the amount of release force that is applied to the flange 206 when the wearable pump 200 is attached to the user may be accounted for and/or adjusted. For example, by moving the center of gravity closer to the user, less release force may be applied to the flange 206, thereby allowing the wearable pump 200 to stay attached to the user. As described above, the vertical plane 224 may be defined as a plane that is parallel to the base 206. Thus, the term “vertical” is relative to the orientation of the wearable pump 200. In some embodiments, the vertical plane 224 may take on any orientation as the base 206 (and thus a plane that is parallel to the base 206) may be tilted in accordance with embodiments of the invention.

A right side view of a wearable pump 200 in a storage orientation 250 in accordance with certain embodiments of the invention is shown in FIG. 2B. In many embodiments, the flat surface 200 allows the wearable pump 201 to rest horizontally in the storage orientation 250. In the storage orientation 250, the flat surface 222 may be horizontal (or substantially horizontal) depending on the orientation of the resting surface 252 (e.g., a table, countertop, refrigerator self, etc.). As described above, there is a difference in the taper angles of the flat surface 222 and the back surface 216. Thus, in many embodiments, the flat surface 222 and the back surface 216 are not parallel. As illustrated in FIG. 2B, in the storage configuration 250, the back surface 216 has a top end 220 that is taller than a bottom end 218 relative to the flat surface 252. In many embodiments, the first and second transition openings 254, 256 between the flange and the milk reservoir, respectively, are located at or near the taller top end 220 such that milk is prevented from spilling out of the milk reservoir 204 in the storage orientation 250. In addition, the flange 202 provides an additional reservoir to keep the milk from spilling. Furthermore, the flange 202 may receive a lid (e.g., lid 150) to seal and contain the milk within the wearable pump 201 when in the storage orientation 250.

Although specific orientations of wearable pumps are discussed above with respect to FIGS. 2A-B, any of a variety of orientations as appropriate to the requirements of a specific application can be used in accordance with embodiments of the invention. Processes for attaching and utilizing wearable pumps in accordance with embodiments of the invention, are discussed further below.

### Processes for Attaching and Utilizing Wearable Pumps

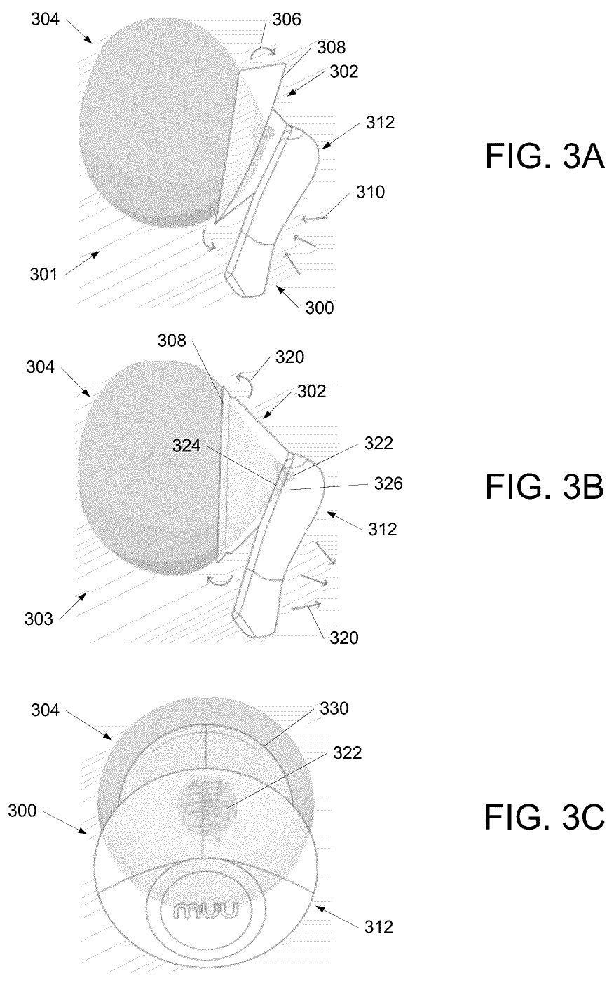

Wearable pumps may be discretely worn and used with various user clothing, including, but not limited to, the user's normal clothes (i.e., clothes that are not specially purposed for breast feeding or breast pumps). A process for attaching 301 a wearable pump 300 to a user in accordance with certain embodiments of the invention is shown in FIGS. 3A-B. In many embodiments, the flange 302 may be attached to the user's breast 304. In reference to FIG. 3A, the flange 302 may be flipped 306 at the base 308, pressure 310 may be applied to the milk reservoir 312 by squeezing the milk reservoir 312 to release air from within the wearable pump 300, and the flipped flange 302 be may be placed on the user's breast 304. In some embodiments, the flange 302 may not be flipped and the user may apply pressure 310 to the milk reservoir 312 and place the flange 302 to the user's breast 304. In some embodiments, the user may first place the flange 302 on the user's breast 304 and then apply pressure 310 to the milk reservoir 312 to allow air to escape.

In reference to FIG. 3B, the process may also include releasing 30 the applied pressure on the milk reservoir 312 thereby applying an attachment suction 320 that attaches the flange 302 to the user's breast 304. In some embodiments, the attachment suction 320 may also re-flip 320 the base 308 of the flange 302 back to a starting position from the flipped 306 position. In many embodiments, the attachment suction 320 may cause the nipple 322 to cross the first and second transition openings 326, 326 of the flange and the milk reservoir. As illustrated in FIG. 3C, once attached, the wearable pump 300 may create a seal 320 around the nipple 322. In various embodiments, the wearable pump 300 may be utilized by applying and releasing a pump suction to the nipple 322 (i.e., a cycle), which expresses milk from the breast 304. In many embodiments, the user may apply and repeat a cycle by squeezing the milk reservoir 312.

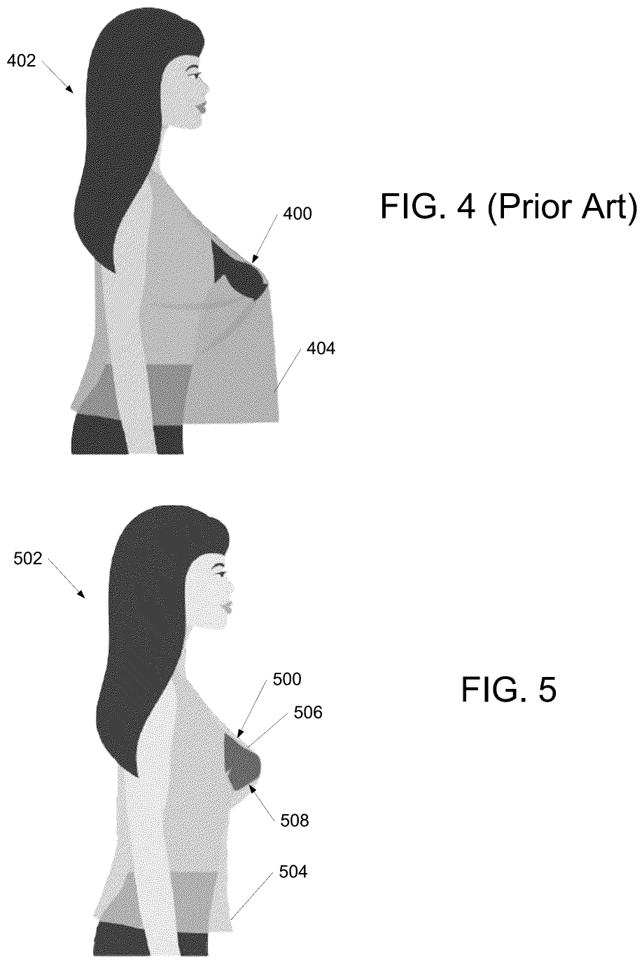

Unlike the current embodiments of the wearable pump, conventional pumps are not optimized for user privacy and wearability. A diagram illustrating a conventional manual pump 400 attached to a user 402 in accordance with the prior art is shown in FIG. 4. As illustrated, the conventional manual pump 400 is not designed to be worn while being utilized. For example, the conventional manual pump 400 causes the user's clothing (e.g., a top 404) to protrude out and is not suited for wearability and/or user privacy. Further, the user 402 may also need to buy specialized clothes for breastfeeding, covers, or clothes that are loose fitting in order to accommodate the conventional manual pump 400.

In contrast to conventional pumps, a diagram illustrating a wearable pump attached to a user in accordance with certain embodiments of the invention is shown in FIG. 5. As described herein, the wearable pump 500 includes a milk reservoir having a dome surface 506 and a flat surface 508 that is angled from the dome surface 506 that allows a user's clothing 504 to drape naturally over the wearable pump 500 for user 502 privacy and wearability. In addition, as further described herein, the configuration of the flange and milk reservoir, including, but not limited to, the truncation plane, rounded first and second front sides, angled back surface, and the concave portion allow for a wider and shorter pump compared to the conventional manual pump resulting in the user's clothing 504 to protrude significantly less. Thus, the present embodiments provide a wearable pump that allows a user's clothing to drape naturally over the wearable pump for user privacy and wearability.

Although specific processes for, and embodiments of, attaching and utilizing wearable pumps are discussed above with respect to FIGS. 3A-5, any of a variety of processes for, and embodiments of, attaching and utilizing wearable pumps as appropriate to the requirements of a specific application can be used in accordance with embodiments of the invention. Wearable pumps with exemplary dimensions in accordance with embodiments of the invention, are discussed further below.

Wearable Pumps with Exemplary Dimensions

As discussed above, wearable pumps may include a flange connected to a milk reservoir optimized for wearability. In many embodiments, the wearable pump may be utilized in a pump orientation and a storage orientation. In the pump orientation, the wearable pump may allow a user's clothing to drape naturally to conceal the wearable pump under the user's clothing. In the storage orientation, the wearable pump may store milk for later use. In some embodiments, the wearable pump may also include a lid configured to connect to the base of the flange and seal the milk in the wearable pump.

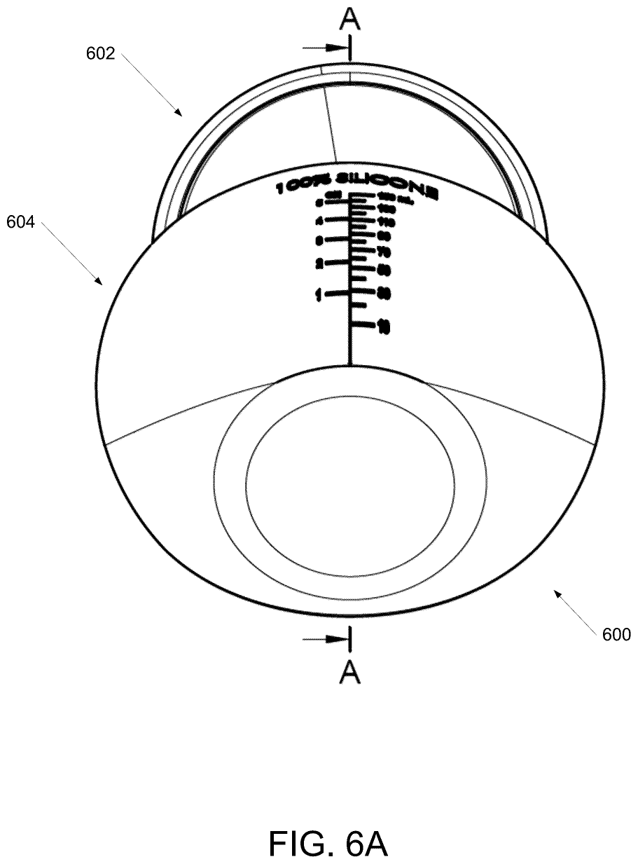

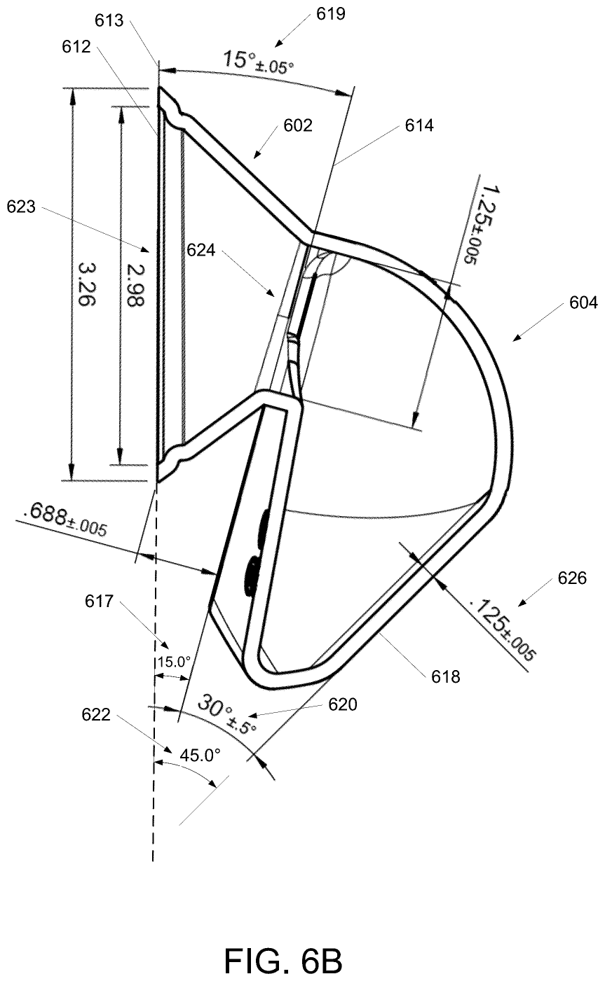

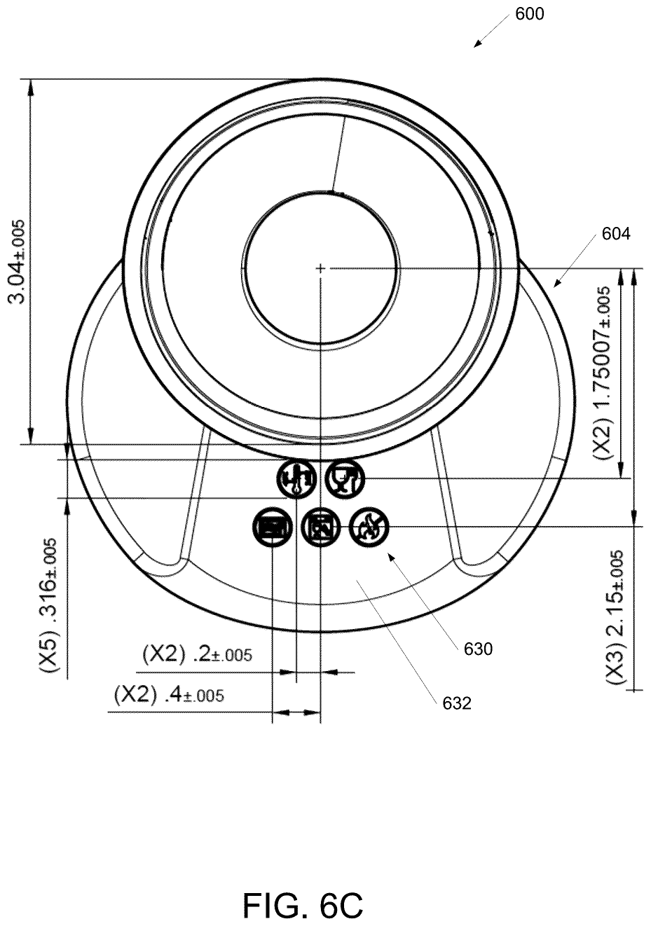

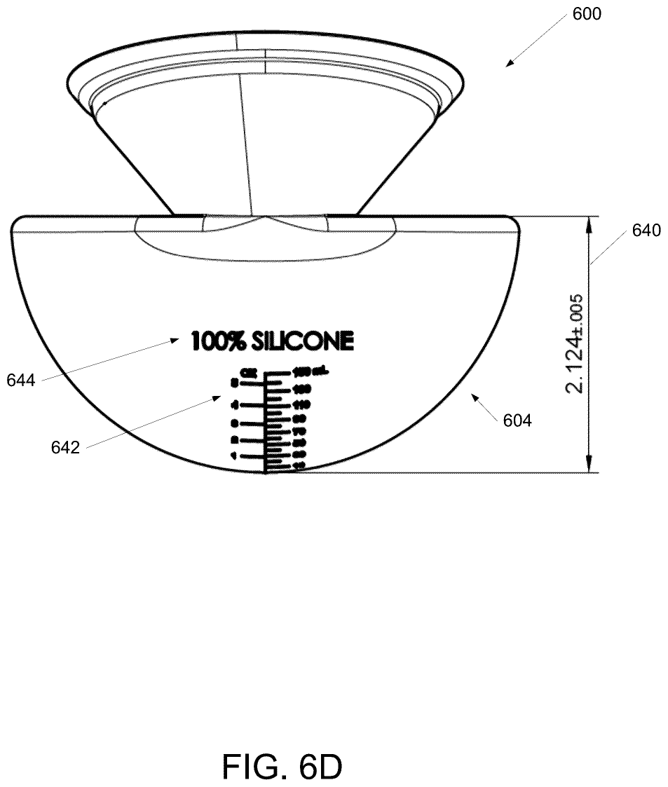

A schematic diagram illustrating a front view of a wearable pump in accordance with certain embodiments of the invention is shown in FIG. 6A. As described above, the wearable pump 600 may include a flange 602 and a milk reservoir 604 where the milk reservoir 604 has a width that is larger than a width of the flange 602. The wider width of the milk reservoir 604 may allow for the wearable pump 600 to be flatter and wear closer to the user resulting in the user's clothing to protrude less than when using convention pumps.

A schematic diagram illustrating a cross-sectional view of a wearable pump along line A-A of FIG. 6A in accordance with certain embodiments of the invention is shown in FIG. 6B. The cross-sectional view has been rotated to show the base 612 of the flange 602 (e.g., a plane defined by the base 612 of the flange 602) as the vertical plane 613. In some embodiments, the truncation plane 614 may be offset by a truncation angle (e.g., 15 degrees) relative to the vertical plane 613. In some embodiments, a back surface 616 of the milk reservoir 604 may be offset by a back surface taper angle 617 (e.g., 15 degrees) relative to the vertical plane 613. In some embodiments, the truncation angle 619 (e.g., 15 degrees) and the back surface taper angle 617 may be the same (as illustrated in FIG. 6B). However, in some embodiments, the truncation angle 619 and the back surface taper angle 617 may be different. In some embodiments, the back surface 616 and the flat surface 618 of the milk reservoir 604 may be offset by an offset angle 620 (e.g., 30 degrees). In some embodiments, the flat surface 618 may be offset relative to the vertical plane 613 by a flat surface taper angle 622 (e.g., 45 degrees). In some embodiments, the flat surface taper angle 622 may be equal to the back surface taper angle 617 (or the truncation angle 619) plus the offset angle 620.

In reference to FIG. 6B, the flange 602 may have a main opening 623 that is larger than the first transition opening. For example, the main opening 623 may be 2.98 inches in diameter and the first transition opening 624 may be 1.25 inches. Further, the flat surface 618 of the milk reservoir 604 may have a thickness 626 (e.g., 0.125 inches) and the flange 602 may have a thickness (e.g., 3.26 inches−2.98 inches=0.28 inches).

A schematic diagram illustrating a back view of a wearable pump 600 in accordance with certain embodiments of the invention is shown in FIG. 6C. In some embodiments, the wearable pump 600 may include information markings 630 such as, but not limited to, dishwasher safety, freezer safety, microwave safety, recycling information, temperature safety ranges, fire safety, etc. In many embodiments, the information marking(s) 600 may be applied in the concave portion 632 of the back surface of the milk reservoir 604.

A schematic diagram illustrating a top view of a wearable pump in accordance with certain embodiments of the invention is shown in FIG. 6D. The wearable pump 600 may have a thickness dimension 640 (e.g., 2.124 inches) that allows the wearable pump 600 to be flatter and wear closer to the user resulting in wearability, as further described above. In some embodiments, the wearable pump 600 may also include measurement markings 642, as further described above. For example, the milk reservoir 604 may include measurement markings 642 in a variety of units (e.g., milliliters, ounces, etc.) that indicate the amount of milk that is in the milk reservoir 604. In many embodiments, the measurement markings 642 may be configured to measure the milk when the wearable pump 600 is in the storage orientation. In some embodiments, the milk reservoir 604 may also include additional markings 644 such as, but not limited to, the material composition of the wearable pump (e.g., 100% silicone).

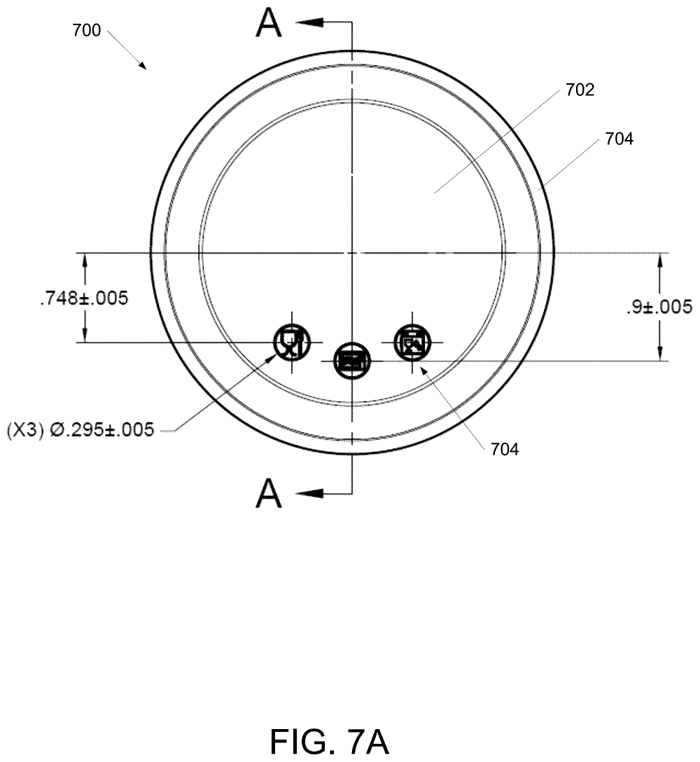

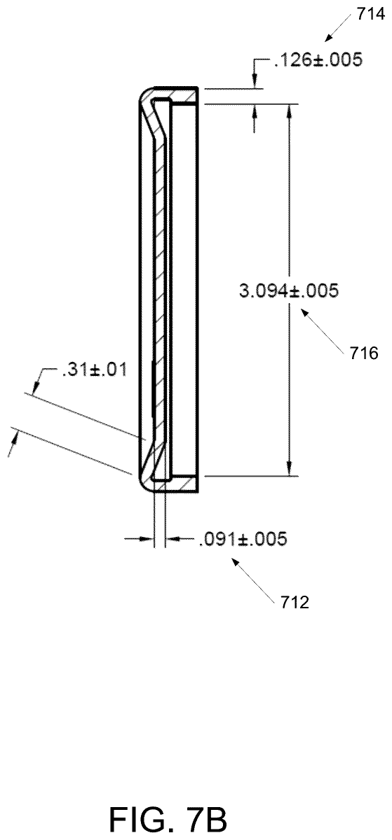

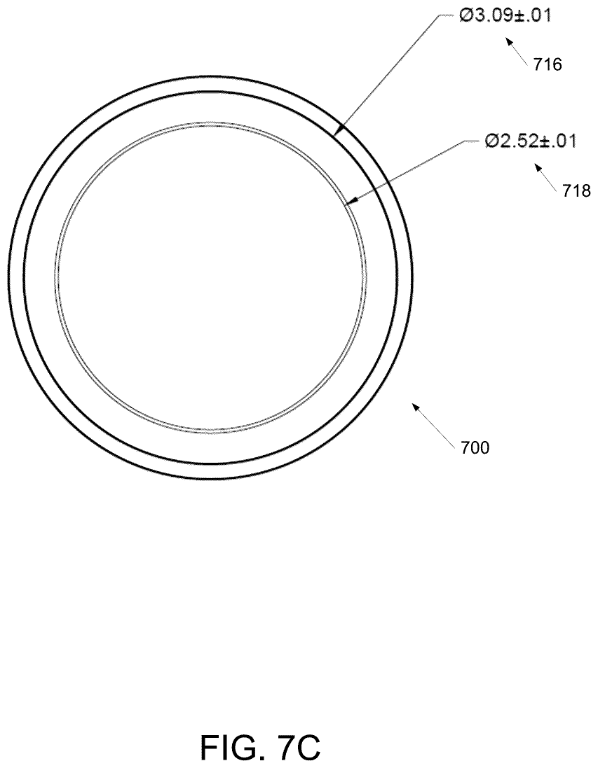

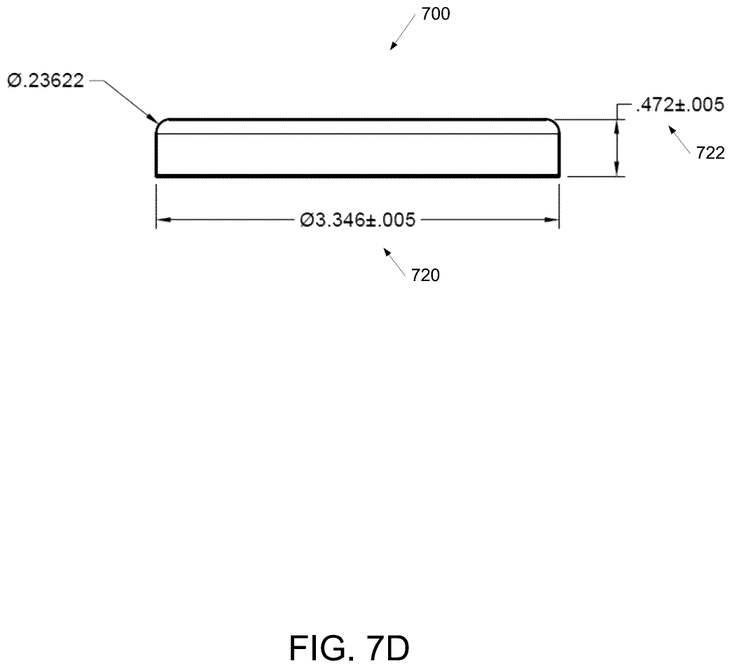

As described above, a lid may connect to the base of the flange and seal the milk in the wearable pump. A schematic diagram illustrating a top view of a lid 700 for a wearable pump in accordance with certain embodiments of the invention is shown in FIG. 7A. The lid 700 may include an inner top portion 702 that includes information markings 704 such as, but not limited to, dishwasher safety, freezer safety, microwave safety, recycling information, temperature safety ranges, fire safety, etc. The lid 700 may also include a rim 704 configured to mate with a flange of a wearable pump, as further described above.

A schematic diagram illustrating a cross-sectional view a lid for a wearable pump along line A-A of FIG. 7A in accordance with certain embodiments of the invention is shown in FIG. 7B. In some embodiments, the inner top portion 702 may have a thickness 712 (e.g., 0.091 inches) and a rim 704 of the lid 700 may have an equal or greater thickness 714 (e.g., 0.126 inches). In some embodiments, the lid 700 may have a first inner diameter 716 (e.g., 3.094 inches).

A schematic diagram illustrating a bottom view of a lid for a wearable pump in accordance with certain embodiments of the invention is shown in FIG. 7C. As described above, the lid 700 may have a first inner diameter 716 (e.g., 3.09 inches) and a second inner diameter 718 (e.g., 2.52 inches). In some embodiments, the first inner diameter 716 may be defined by the rim 704 that may be tapered.

A schematic diagram illustrating a side view of a lid for a wearable pump in accordance with certain embodiments of the invention is shown in FIG. 7D. The lid 700 may have an external diameter 720 (e.g., 3.346 inches), a height 722 (e.g., 0.472 inches), and an inner top portion external diameter (e.g., 0.23622 inches).

Although specific dimensions of wearable pumps and lids are discussed above with respect to FIGS. 6A-7D, any of a dimensions and configurations of wearable pumps as appropriate to the requirements of a specific application can be used in accordance with embodiments of the invention. While the above description contains many specific embodiments of the invention, these should not be construed as limitations on the scope of the invention, but rather as an example of one embodiment thereof. It is therefore to be understood that the present invention may be practiced otherwise than specifically described, without departing from the scope and spirit of the present invention. Thus, embodiments of the present invention should be considered in all respects as illustrative and not restrictive.

What is claimed is:

1. A wearable breast pump optimized for user privacy and wearability, the wearable breast pump comprising:
a flange that is hollow, wherein the flange comprises:
a main opening for receiving and attaching to a user's breast; and
a first transition opening that allows passage between the flange and a milk reservoir;
the milk reservoir that is hollow and connected to the flange, the milk reservoir comprising:
a second transition opening that allows passage between the flange and the milk reservoir;
a dome surface that drapes a user's clothing when the wearable breast pump is in a pump orientation; and
a flat surface connected to the dome surface, wherein the flat surface allows the wearable breast pump to rest horizontally in a storage orientation.

2. The wearable breast pump of claim 1, wherein the flange comprises a base that is flat and an apex located opposite the base, wherein the flange tapers from the base towards the apex.

3. The wearable breast pump of claim 2, wherein the flange is a three-dimensional shaped cone with the apex sliced by a truncation plane.

4. The wearable breast pump of claim 3, wherein the truncation plane is offset relative to a vertical plane defined along the base.

5. The wearable breast pump of claim 4, wherein the truncation plane is offset relative to the vertical plane by 15 degrees.

6. The wearable breast pump of claim 5, wherein the milk reservoir is attached to the flange along the truncation plane.

7. The wearable breast pump of claim 4, wherein the milk reservoir further comprises a back surface that runs along the truncation plane.

8. The wearable breast pump of claim 7, wherein the back surface comprises first and second back sides are flat.

9. The wearable breast pump of claim 8, wherein the back surface further comprises a concave portion located between the first and second back sides, wherein the concave portion allows the wearable pump to wear closer to the user's body.

10. The wearable breast pump of claim 7, wherein the back surface tapers in towards the user at a back surface taper angle relative to the vertical plane.

11. The wearable breast pump of claim 10, wherein the back surface taper angle is equal to 15 degrees.

12. The wearable breast pump of claim 10, wherein the flat surface of the milk reservoir tapers towards the user at a flat surface taper angle relative to the vertical plane.

13. The wearable breast pump of claim 12, wherein the flat surface taper angle is greater than the back surface taper angle.

14. The wearable breast pump of claim 13, wherein the flat surface taper angle is at least double the back surface taper angle.

15. The wearable breast pump of claim 13, wherein the flat surface taper angel is 45 degrees.

16. The wearable breast pump of claim 1, the milk reservoir has a milk reservoir diameter measured at a widest portion of the milk reservoir and the flange has a flange diameter measured at a widest portion of the flange, wherein the milk reservoir diameter is greater than the flange diameter.

17. The wearable breast pump of claim 1, wherein the flat surface of the milk reservoir is circular.

18. The wearable breast pump of claim 17, wherein the milk reservoir further comprises a first front side and a second front side, wherein the first and second front sides are curved relative to the flat surface.

19. The wearable breast pump of claim 1 further comprising a lid that connects to the base of the flange and seals milk within the wearable pump when the wearable pump is in the storage orientation.

20. The wearable breast pump of claim 1, wherein the milk reservoir further comprises measurement markings that indicate an amount of milk that is within the milk reservoir when the wearable pump is in the storage orientation.
